(19) United States
(12) Patent Application Publication
(10) Pub. No.: US 2011/0059977 A1
(43) Pub. Date: Mar. 10, 2011

(54) USE OF CANTHIN-6-ONE AND ITS ANALOGS IN THE TREATMENT OF MYCOBACTERIA-LINKED PATHOLOGIES ( amended

(75) Inventors: Alain Robert Francois Maxime Fournet, Ossages (FR); Delphine Lagoutte, Fontenay-Aux-Roses (FR); Erwan Poupon, Antony (FR); Flor Soriano-Agaton, Mexico (MX)

(73) Assignee: INSTITUT DE RECHERCHE POUR LE DEVELOPPEMENT (I.R.D.), Paris (FR)

(21) Appl. No.: 12/294,668

(22) PCT Filed: Mar. 22, 2007
(86) PCT No.: PCT/FR2007/000486

§ 371 Date: Dec. 30, 2008

(30) Foreign Application Priority Data
Mar. 28, 2006 (FR) 0602677

(51) Int. Cl. A61K 31/4985; C07D 471/16; A61K 31/4375; C07D 471/22; A61K 31/4745; A61P 31/06

(52) U.S. Cl. 514/250; 546/68; 514/288; 544/342; 546/51; 514/283

(57) ABSTRACT

The present invention relates to the use, for the preparation of a medicament intended for the treatment or the prevention of pathologies linked to, or caused by mycobacteria, of at least one of the compounds of the following formula (I): in which B represents in particular a nitrogen atom, and R1, R2, R3, R4, R5, R6, R7 and R8 represent in particular a hydrogen atom.

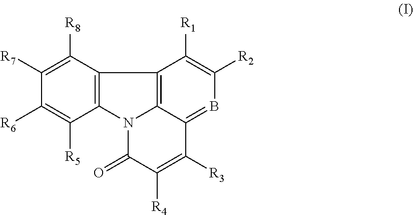

This invention relates to the use of canthin-6-one and its analogs in the preparation of a drug for the treatment or prevention of pathologies linked to, or caused by mycobacteria, particularly tuberculosis.

Canthin-6-one is a known compound which has been isolated from plants such as: Ailanthus altissima (Simaroubaceae) by Ohmoto et al., Chem. Pharm. Bull., 1976, 24, 1532-1536; Brucea antidysenterica (Simaroubaceae) by Fukamiya et al., Planta Med., 1987, 53, 140-143; Eurycoma harmandiana (Simaroubaceae) by Kachanapoom et al., Phytochemistry, 2001, 56, 383-386; Peganum nigellastrum (Zygophyllaceae) by Ma et al., Phytochemistry, 2000, 53, 1075-1078.

Canthin-6-one and some of its derivatives show interesting pharmacological activities, and notably an antifungal activity (Thouvenel et al., 2003; Soriano-Agatón et al., 2005), and also a trypanocidal activity in Chagas' disease (WO 2004/050092).

Mycobacteria, among other bacteria, have been thoroughly studied because they are responsible for two serious diseases: tuberculosis and leprosy.

Tuberculosis is an infectious, contagious and endemic disease, with a highly marked respiratory tropism, due to Mycobacterium tuberculosis (or Koch's bacillus). Mycobacterium tuberculosis belongs to the genus mycobacteria, and so does the leprosy bacillus (Mycobacterium leprae).

It is thought that tuberculosis kills some two million people each year (WHO, 2006). Moreover tuberculosis is more and more often associated with infections by the HIV virus (responsible for ca. 13% of deaths by AIDS worldwide); hence the importance of an anti-tuberculosis treatment for immunosuppressed patients, and above all AIDS patients.

Pulmonary tuberculosis is the most frequent variant, but other parts of the body may be infected (kidney, joints, genital organs, pericardium, brain . . . ).

Tuberculosis contamination is essentially due to the release of bacilli into the atmosphere, via droplets which are suspended in the air. Inhalation of a small number of droplets is enough for the infection of one individual. Transmission by food (ingestion of meat or milk which have been contaminated by Mycobacterium bovis) has all but disappeared.

The treatment of any form of tuberculosis (respiratory tract tuberculosis, bone and joints tuberculosis) rests upon the regular uptake of antibiotics. With any patient, a manifest primary infection with radiological or systemic signs must be considered as a tuberculosis and treated as such.

The standard treatment rests on the daily uptake, during 6 months, of isoniazide and rifampicine, together with the administration of pyrazinamide and ethambutol during the first two months (Working Group of the French High Council of Public Hygiene). The first aim of this polytherapy is to exert an influence upon the various bacilli populations, and to obtain a cure in six months' time. The second aim is to prevent the selection of mutants which are resistant, and which could cause a resistant bacilli relapse.

However to this day the standard treatment is not always enough to prevent the appearance of resistant strains.

One of the aims of the invention is to provide a new class of compounds which are active for the treatment of pathologies which are associated with or caused by mycobacteriae.

One of the aims of the invention is to provide new drugs against tuberculosis with pharmacological properties whose efficiency is comparable to that of already used drugs, but which make it possible to remedy the emergence of strains which are resistant to the drugs in use.

One of the aims of the invention is to provide new drugs against tuberculosis which are active with immunosuppressed patients.

In this invention it has been found surprisingly that canthin-6-one and its derivatives have antimycobacterial properties.

This invention relates to the use, in the preparation of a drug for the treatment or prevention of pathologies which are linked to or caused by mycobacteria, of at least one compound having the following Formula (I):

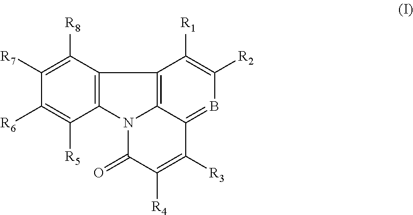

in which:
B represents:
   a nitrogen atom, or
   a N-oxide N⁺—O⁻, group, or
   a group having the formula N⁺—R ou

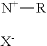

R representing an alkyl group, which is linear, branched or cyclic, saturated or unsaturated, comprising from 1 to 12 carbon atoms, preferably from 1 to 6 carbon atoms, which may be substituted, for example by at least one halogen atom, and X⁻ represents an anion which may be chosen among mineral or organic anions,
R₁, R₂, R₃, R₄, R₅, R₆, R₇ and R₈ represent independently:
   a hydrogen atom,
   an alkyl group which is linear, branched or cyclic, saturated or unsaturated, comprising from 1 to 12 carbon atoms, preferably from 1 to 6 carbon atoms,
   a halogen atom chosen among chlorine, fluorine, bromine and iodine,
   a halogenoalkyl group, in which the alkyl chain is linear, branched or cyclic, saturated or unsaturated, comprising from 1 to 12 carbon atoms, preferably from 1 to 6 carbon atoms, and in which the halogen atom(s) are chosen among fluorine, chlorine, bromine or iodine,
   a hydroxyl group,
   a nitro —NO group,
   a cyano —CN group,
   a —SH group,
   a carboxylic acid —COOH group,
   an amide —CONH₂ group,
   an amine —NH₂ group,
   an alcoxy —ORₐ, group, wherein Rₐ represents an alkyl group, which may be linear, branched or cyclic, saturated or unsaturated, comprising from 1 to 12 carbon atoms, preferably from 1 to 6 carbon atoms,
   an alkylester —COOR_{b} group, wherein R_{b} represents an alkyl group, which may be linear, branched or cyclic, saturated or unsaturated, comprising from 1 to 12 carbon atoms, preferably from 1 to 6 carbon atoms,
   a secondary (—NHCOR_{c}) or tertiary
   (—N(COR_{d})COR_{c}) alkylamide group, wherein R_{c} and R_{d} independently represent an alkyl group, which may be linear, branched or cyclic, saturated or unsaturated, comprising from 1 to 12 carbon atoms, preferably from 1 to 6 carbon atoms,
   a secondary (—NHRₑ) or tertiary (—NRₑR_{f}) alkylamine group, wherein Rₑ and R_{f} independently represent an alkyl group which may be linear, branched or cyclic, saturated or unsaturated, comprising from 1 to 12 carbon atoms, preferably from 1 to 6 carbon atoms,
   an alkylthio (—SR_{g}) group, wherein R_{g} represents an alkyl group, which may be linear, branched or cyclic, saturated or unsaturated, comprising from 1 to 12 carbon atoms, preferably from 1 to 6 carbon atoms,
   a C₂-C₆ heterocyclic group, comprising from 1 to 4 heteroatoms chosen among sulphur, nitrogen and oxygen,
   a —SO₂—NRₕRᵢ, group or a —NRₕ—SO₂—Rᵢ group, wherein Rₕ-Rᵢ, independently represent an alkyl group, which may be linear, branched or cyclic, saturated or unsaturated, comprising from 1 to 12 carbon atoms, preferably from 1 to 6 carbon atoms,
the groups R₁, R₂, R₃, R₄, R₅, R₆, R₇ and R₈ may also form the following intramolecular cyclisations:
   1/ cyclisation between R¹ and R² and/or
   2/ cyclisation between R³ and R⁴ and/or
   3/ cyclisation between R⁵ and R⁶ and/or
   4/ cyclisation between R⁶ and R⁷ and/or
   5/ cyclisation between R⁷ and R⁸,
   6/ cyclisation between R³ and B, notably when B represents N⁺—R,
   7/ cyclisation between R² and B, notably when B represents N⁺—R,

said thus formed cycles being notably aromatic cycles comprising from 5 to 30 carbon atoms, and which may contain at least one heteroatom chosen among: O, N, S, the aromatic cycles being for example chosen among benzene, naphthalene, pyridine, pyrrole, thiophene, furan, pyrazine,

wherein said cycles may be substituted by
a hydrogen atom,
a linear, branched or cyclic, saturated or unsaturated alkyl group, comprising from 1 to 12 carbon atoms, preferably from 1 to 6 carbon atoms,
a halogen atom chosen among chlorine, fluorine, bromine and iodine,
a halogenoalkyl group, wherein the alkyl chain is linear, branched or cyclic, saturated or unsaturated, comprising from 1 to 12 carbon atoms, preferably from 1 to 6 carbon atoms, and wherein the halogen atom(s) are chosen among fluorine, chlorine, bromine and iodine,
a hydroxyl group,
a nitro —NO group,
a cyano —CN group,
a —SH group, —
a carboxylic acid —COOH group,
a —CONH₂ group,
an amine —NH₂ group,
a —ORₐ group, wherein Rₐ represents an alkyl group, which may be linear, branched or cyclic, saturated or unsaturated, comprising from 1 to 12 carbon atoms, preferably from 1 to 6 carbon atoms,
an alkylester —COOR_{b} group, wherein R_{b} represents an alkyl group, which may be linear, branched or cyclic, saturated or unsaturated, comprising from 1 to 12 carbon atoms, preferably from 1 to 6 carbon atoms,
a secondary (—NHCOR_{c}) or tertiary
(—N(COR_{d})COR_{c}) alkylamide group, wherein R_{c} and R_{d} independently represent an alkyl group, which may be linear, branched or cyclic, saturated or unsaturated, comprising from 1 to 12 carbon atoms, preferably from 1 to 6 carbon atoms,
a secondary (—NHRₑ) or tertiary (—NRₑR_{f}) alkylamine group, wherein Rₑ and R_{f} independently represent an alkyl group, which may be linear, branched or cyclic, saturated or unsaturated, comprising from 1 to 12 carbon atoms, preferably from 1 to 6 carbon atoms,
an alkylthio (—SR_{g}) group, wherein R_{g} represents an alkyl group, which may be linear, branched or cyclic, saturated or unsaturated, comprising from 1 to 12 carbon atoms, preferably from 1 to 6 carbon atoms,
a C₂-C₆ heterocyclic group, comprising from 1 to 4 heteroatoms chosen among sulphur, nitrogen and oxygen,
a —SO₂—NRₕRᵢ group or a —NRₕ—SO₂—Rᵢ group, wherein Rₕ and Rᵢ independently represent an alkyl group, which may be linear, branched or cyclic, saturated or unsaturated, comprising from 1 to 12 carbon atoms, preferably from 1 to 6 carbon atoms.

Canthin-6-one corresponds to Formula (I) wherein the substituents R₁-R₈ represent a hydrogen atom and B represent nitrogen.

By mycobacteria is meant all species of the genus Mycobacterium, classified in the order of Actinomycetales, whose common characteristic is to be detected by Ziehl-Neelsen staining, and to be acid-alcohol-resistant (for example Timo Ulrichs et al. J. Pathol. 2005, 205:633-640 <<Modified immunohistological staining allows detection of Ziehl-Neelsen-negative Mycobacterium tuberculosis organisms and their precise localization in human tissue>>).

According to an advantageous embodiment the mycobacteria as included in the invention belong to the group comprising Mycobacterium tuberculosis, Mycobacterium bovis, Mycobacterium smegmatis, Mycobacterium africanum, Mycobacterium leprae, Mycobacterium kansasii, Mycobacterium xenopi, Mycobacterium avium intracellulaire, Mycobacterium scrofulaceum, Mycobacterium marinum, Mycobacterium fortuitum, Mycobacterium chelonei, Mycobacterium ulcerans and Mycobacterium abcessus.

According to an advantageous embodiment the invention relates to the use of the hereabove Formula (I) compounds, in the preparation of a drug for the treatment or prevention of pathologies which are linked to or caused by Mycobacterium tuberculosis or Mycobacterium bovis.

Advantageously the invention relates to the use of a hereabove defined Formula (I) compound, in the preparation of a drug for the treatment or the prevention of the following pathologies:

Tuberculosis, and particularly
Tuberculosis of the respiratory tract (pulmonary tuberculosis, tuberculous lymphadenitis, larynx, trachea and bronchi tuberculosis, tuberculous pleurisy and tuberculous primary infection of the respiratory tract),
Tuberculosis of the nervous system (tuberculous meningitis, tuberculous polyneuritis, tuberculous myelitis),
Tuberculosis of the bone and joints,
Tuberculosis of the urogenital tract,
Peripheral tuberculous adenopathy,
Tuberculosis of the intestine, the peritoneum and the mesenteric glands
Tuberculosis of the skin and the subcutaneous cellular tissue
Tuberculosis of the eye and ear
Tuberculosis of the adrenal glands
Acute miliary tuberculosis

Buruli's ulcer,

Nosocomial infections,

Leprosy,

Cutaneous infections,

Soft tissue infections,

Osteomyelites,

Localized abscesses,

Lipoid pneumoniae.

According to another advantageous embodiment, in the Formula (I) compounds wherein B represents

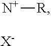

X⁻ is chosen among Cl⁻, Br⁻, I⁻, S⁻, PO₃⁻, NO₃⁻,

acetate, oxalate, tartrate, succinate, maleate, fumarate, gluconate, citrate, malate, ascorbate, benzoate.

According to an advantageous embodiment the invention relates to the use, such as hereabove defined, of Formula (I) compounds wherein B represents a nitrogen atom and the compounds correspond to the following Formula (II):

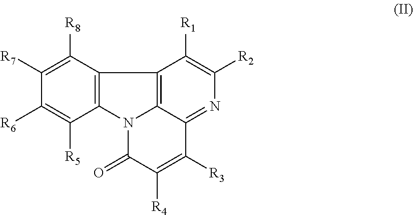

According to an advantageous embodiment, the invention relates to the use, as hereabove defined, of Formula (I) compounds wherein B represents a N-oxide NO group, and the compounds correspond to the following Formula (III):

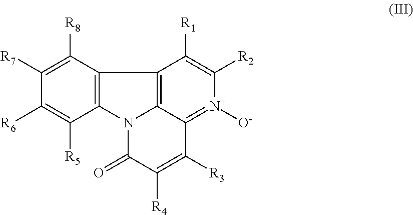

According to an advantageous embodiment, the invention relates to the use, as hereabove defined, of Formula (I) compounds, wherein B represents a group having the Formula,

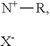

X⁻ and R being such as hereabove defined, and the compounds correspond to the following Formula (IV):

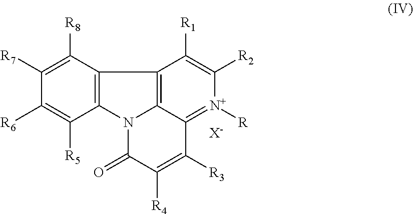

R notably represents a methyl or ethyl group, for instance substituted with at least one halogen atom such as F, Cl, Br, or I.

According to an advantageous embodiment, the invention relates to the use, as hereabove defined, of Formula (I) compounds, wherein R₃ and R₄ form a benzene cycle between them, and the compounds correspond to the following Formula (I-1):

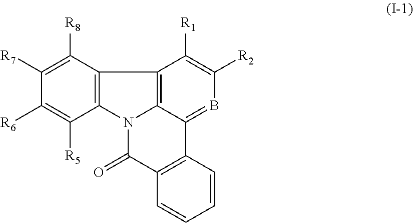

According to an advantageous embodiment of the invention, the Formula (I) compounds are chosen among:

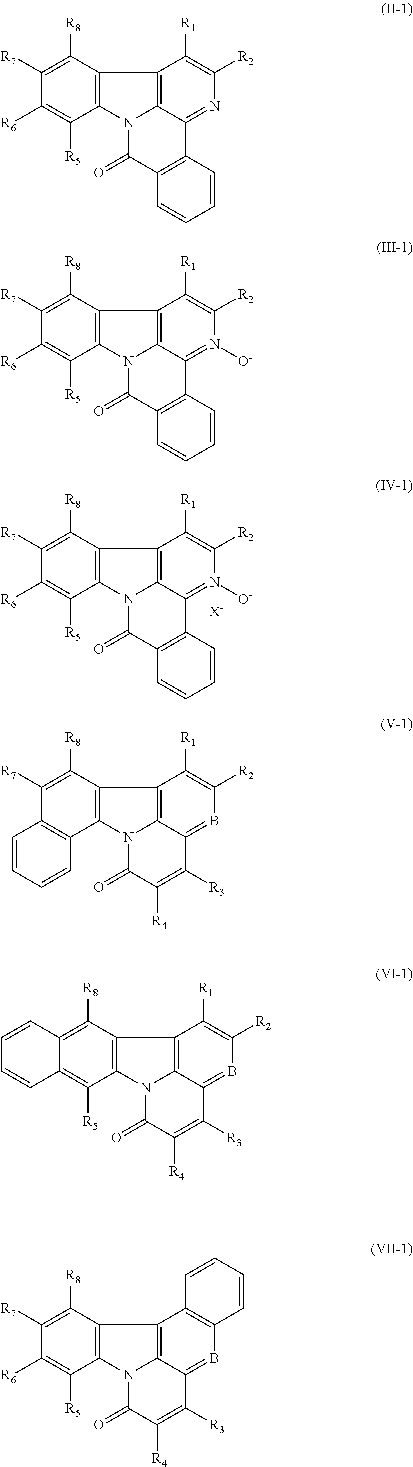

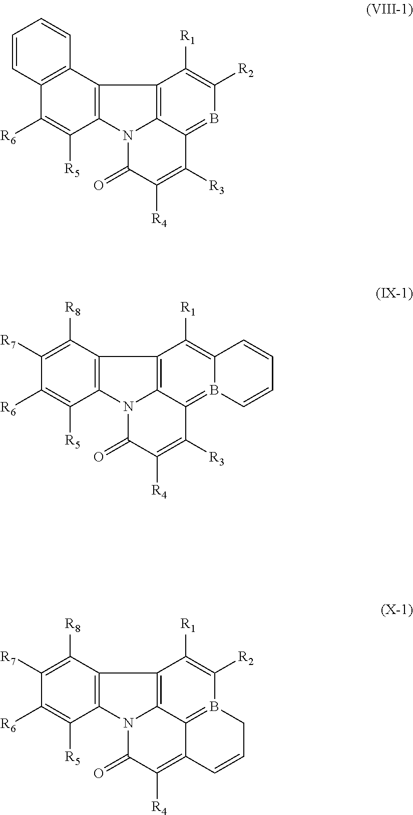

wherein R₁, R₈, B and X⁻ have the hereabove given meanings.

According to another advantageous embodiment, the Formula (I) compounds as used are such that at least one of radicals R₅-R₈, and notably R₆, represents a halogen atom, notably a fluorine atom.

According to another advantageous embodiment of the invention, the Formula (I) compound which is used is canthin-6-one, which corresponds to the following formula;

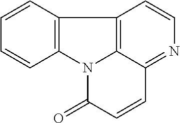

According to an advantageous embodiment of the invention the Formula (I) compounds which are used are chosen among one of the following compounds:

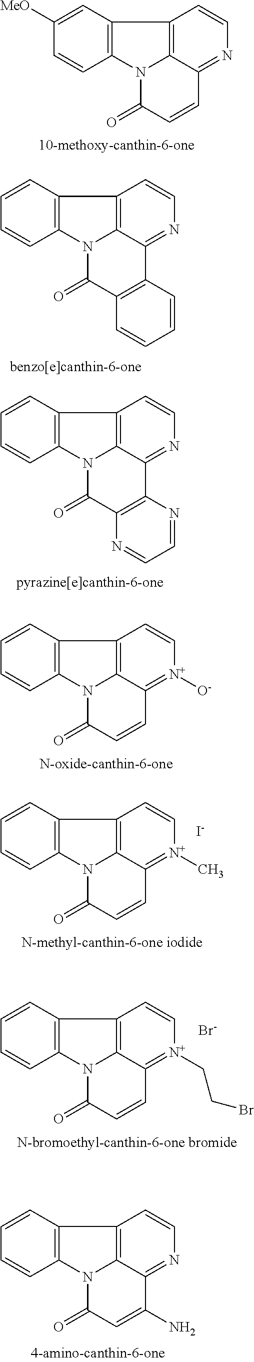

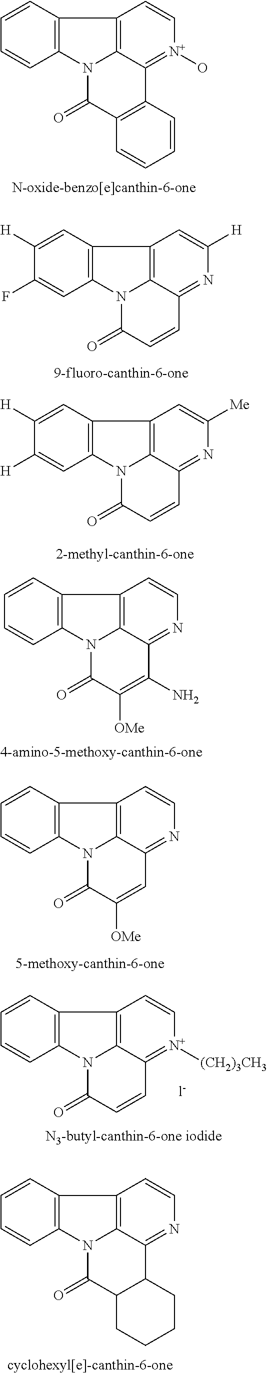

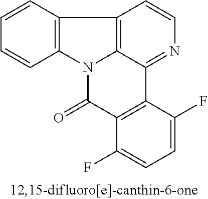

The above-defined Formula (I) compounds are advantageously delivered at a dose from ca, 0.01 mg/kg/day to ca. 100 mg/kg/day, preferably of ca. 0.1-50 mg/kg/day, and advantageously from ca. 1 to ca. 20 mg/kg/day.

No toxicity has been observed in the various experiments for determining the lethal dose (LD50). Assays have been made with mice by intraperitoneally delivering a dose of 200 mg/kg/day of a Formula (I) compound. All tested mice survived. According to an advantageous embodiment of the invention the Formula (I) compounds as used in the invention are delivered orally.

The invention also relates to the compound N-oxide-benzo[e]canthin-6-one, corresponding to the following formula:

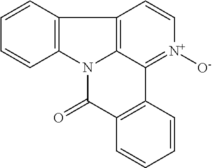

This compound is a new compound.

The invention also relates to a pharmaceutical composition comprising as an active substance the compound N-oxide-benzo[e]canthin-6-one, corresponding to the following formula:

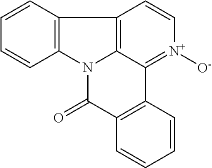

in association with a pharmaceutically acceptable excipient.

The compounds as used in the invention may be prepared according to a process as described in Patent Application WO 2004/050092. In this application canthin-6-one has been isolated from plant extracts which contain it, and notably from the bark of the trunk of a Rutacea identified as Zanthoxylum chiloperone var. angustifolium.

Canthin-6-one and its derivatives may be prepared by total chemical synthesis or hemisynthesis, as described in <<Soriano-Agatón et al., 2005; Journal of Natural Products, Volume 68, Number 11, November, 2005>>.

The following examples are given for illustrating in greater detail the various aspects of the invention, but they must not be construed as being forms which limit the invention.

### EXAMPLE 1

### Synthesis of canthin-6-one and its Analogs

Canthin-6-one and its analogs are obtained by total synthesis. A rapid access pathway to the general skeleton, allowing pharmacomodulations, has been developed.

The following Scheme follows this strategy for obtaining canthin-6-one

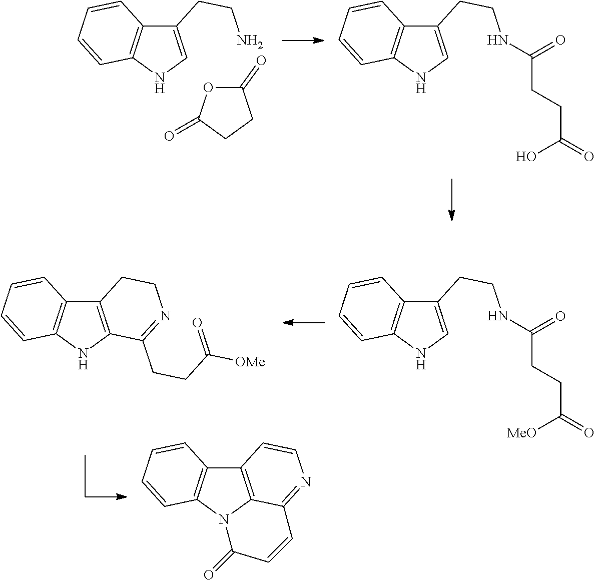

### 1-Synthesis of canthin-6-one from Tryptamine

### 4-[2-(1-H-3-indolyl)ethylcarbamoyl]-butanoic acid

In CH₂Cl₂ (50 tryptamine (0.01 mole) is dissolved and succinic anhydride (1.1 eq.) is gradually added. This solution is agitated during 18 hours at room temperature, then concentrated under reduced pressure. The obtained solid is triturated with a minimum amount of CH₂Cl₂, filtrated and dried (>97%).

### Methyl 4-[2-(1H-3-indolyl)ethylcarbamoyl]-butanoate

4-[2-(1-H-3-indolyl)ethylcarbamoyl]-butanoic acid (0.008 mole) is dissolved in methanol (50 mL). Amberlyst H-15® resin (20% m/m) is added and the mixture is refluxed during 18 hours. After elimination of the resin by filtration and concentration under reduced pressure the obtained solid is triturated in ethly ether, then filtrated and dried (>97%).

### Methyl 4-(3-4-dihydro-9H-β-carboline-1-yl)-butanoate

Methyl 4-[2-(1H-3-indolyl)ethylcarbamoyl]-butanoate (0.90 mmole) is dissolved in benzene (10 mL). POCl₃ (3 eq.) is added dropwise at 5° C. The mixture is refluxed during 1 hour, then concentrated under reduced pressure and used as such in the next step.

### Canthin-6-one

In CH₂Cl₂ (25 mL) methyl 4-(3-4-dihydro-9H-β-carboline-1-yl)-butanoate (0.50 mmole) is dissolved and DBU (diazabicycloundecene, 3 eq.) is added. This solution is agitated during 18 hours at room temperature, then washed with a saturated aqueous NaHCO₃ solution. The organic phase is dried (Na₂SO₄), then concentrated under reduced pressure. Canthin-6-one is purified by a silica gel column chromatography (elution:CH₂Cl₂/MeOH 99:1, 60% two steps).

### 2-Synthesis of canthin-6-one Derivatives

Canthin-6-one analogs may be synthesized by using techniques such as described in <<Soriano-Agatón et al., 2005; Journal of Natural Products, Volume 68, Number 11, November, 2005>>.

The compound N-oxide-benzo[e]canthin-6-one is prepared according to the following protocol:

Benzo[e]canthin-6-one (50 mg-0.2 mmole) is dissolved in CH₂Cl₂ (25 mL), then m-chloroperbenzoic acid (3 eq.) is added. This solution is agitated during 18 hours at room temperature. Water (25 mL) is added and the bi-phase medium is agitated during 1 hr, The organic phase is then washed with a saturated aqueous NaHCO₃ solution (3×20 mL), then dried (Na₂SO₄) and concentrated under reduced pressure. The obtained product is purified by silica gel column chromatography (elution:CH₂Cl₂/MeOH 9:1) to give the N-oxide-benzo[e]canthin-6-one in the form of a powder (40 mg-70%).

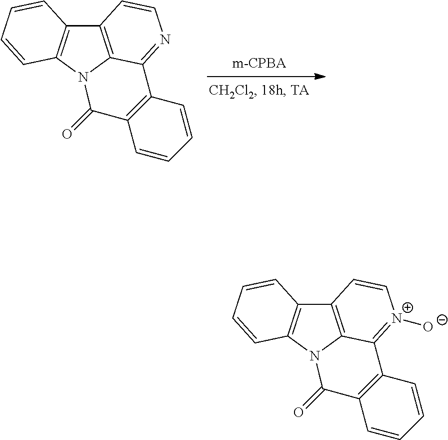

### 3-Analytic Data for canthin-6-one and its Analogs

Canthin-6-one:

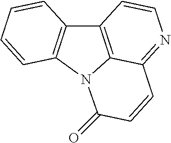

microcrystalline yellow powder
melting point: 161-163° C.
IR υₘₐₓ cm⁻¹: 1,673 (-
MS (IE): m/z [M + Na]⁺ 243
MSHR (IC): for C₁₄H₈N₂ONa
[M + Na]⁺ 243.0534, found: m/z 243.0532
R_{f}: 0.6 (CH₂Cl₂/MeOH 9:1)
MNR ¹H (400 MHz, CDCl₃):

| δ (ppm) | multiplicity | coupling ⁿJ (Hz) | integration | attribution |
|---|---|---|---|---|
| 6.87 | d | ³J 9.8 Hz | 1H | H-5 |
| 7.41 | t | ³J 8.0 Hz | 1H | H-10 |
| 7.58 | t | ³J 8.0 Hz | 1H | H-9 |
| 7.80 | d | ³J 5.0 Hz | 1H | H-1 |
| 7.90 | d | ³J 9.8 Hz | 1H | H-4 |
| 7.94 | d | ³J 8.0 Hz | 1H | H-11 |
| 8.50 | d | ³J 8.0 Hz | 1H | H-8 |
| 8.70 | d | ³J 5.0 Hz | 1H | H-2 |

MNR ¹³C (100 MHz, CDCl₃): 116.1 (C-1); 117.0 (C-8); 122.4
(C-11); 124.1 (C-11a); 125.4 (C-10); 128.7 (C-5); 129.9 (C-11b);
130.6 (C-9); 131.7 (C-3b); 135.9 (C-3a); 139.1 (C-7a); 139.3 (C-4);
145.6 (C-2); 159.2 (C-6).
10-methoxy-canthin-6-one:

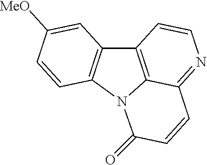

microcrystalline yellow powder
Melting point: 203-205° C.
IR υₘₐₓ cm⁻¹: 1,673 (—C═O)
MS (IC): m/z [M + H]⁺ 251
MSHR (IC): for C₁₅H₁₁N₂O₂
[M + H]⁺ 251.0821, found: m/z 251.0825
R_{f}: 0.6 (CH₂Cl₂/MeOH 9:1)
RMN ¹H (400 MHz, CDCl₃):

| δ (ppm) | multiplicity | coupling ⁿJ (Hz) | integration | attribution |
|---|---|---|---|---|
| 3.96 | s | | 3H | —OMe |
| 6.98 | d | ³J 9.8 Hz | 1H | H-5 |
| 7.27 | dd | ³J 9.0 Hz. ⁴J 2.1 Hz | 1H | H-9 |
| 7.57 | d | ⁴J 2.1 Hz | 1H | H-11 |
| 7.96 | d | ³J 5.0 Hz | 1H | H-1 |
| 8.05 | d | ³J 9.8 Hz | 1H | H-4 |
| 8.55 | d | ³J 9.0 Hz | 1H | H-8 |
| 8.81 | d | ³J 5.0 Hz | 1H | H-2 |

MNR ¹³C (100 MHz, CDCl₃): 56.0 (—OMe); 106.7 (C-11); 116.4
(C-1); 118.1 (C-8); 118.3 (C-9); 125.8 (C-11a); 129.4 (C-5); 131.0
(C-11b); 132.5 (C-3b); 133.8 (C-7a); 136.0 (C-3a); 138.7 (C-4);
145.0 (C-2); 158.1 (C-10); 159.2 (C-6).
Benzo[e]canthin-6-one:

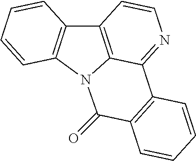

microcrystalline white powder
Melting point: 228-230° C.
IR υₘₐₓ cm⁻¹: 1,681 (—C═O)
MS (IE): m/z [M + H]⁺ 271
MSHR (IC): for C₁₈H₁₁N₂O₂
[M + H]⁺ 271.0871, found: m/z 271.2073
R_{f}: 0.6 (CH₂Cl₂/MeOH 9:1)
MNR ¹H (400 MHz, CDCl₃):

| δ (ppm) | multiplicity | coupling ⁿJ (Hz) | integration | attribution |
|---|---|---|---|---|
| 7.51 | t | ³J 8.0 Hz | 1H | H-10 |
| 7-68-7.76 | m | | 2H | H-14, H-9, |
| 7.87 | t | ³J 8.0 Hz | 1H | H-13 |
| 7.91 | d | ³J 5.0 Hz | 1H | H-1 |
| 8.10 | d | ³J 8.0 Hz | 1H | H-11 |
| 8.61 | d | ³J 8.0 Hz | 1H | H-15 |
| 8.75 | m | | 2H | H-12. H-8 |
| 8.81 | d | ³J 5.0 Hz | 1H | H-2 |

MNR ¹³C (100 MHz, CDCl₃): 115.1 (C-1); 117.5 (C-8); 122.4
(C-11); 123.5 (C-12); 124.9 (C-11a); 125.3 (C-10); 129.2 (C-15);
129.5 (C-3a); 130.0 (C-14); 130.4 (C-3b); 130.5 (C-11b); 130.6
(C-9); 133.5 (C-13); 134.7 (C-4); 136.0 (C-5); 139.4 (C-7a); 144.9
(C-2); 159.4 (C-6).
Pyrazine[e]canthin-6-one:

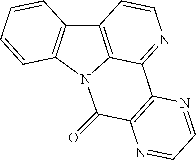

microcrystalline brown
Melting point: 250-253° C.
IR υₘₐₓ cm⁻¹: 1,699 (—C═O)
MS (IE): m/z [M + Na]⁺ 295
MSHR (IC): for C₁₆H₈N₄ONa
[M + Na]⁺ 295.0596, found: m/z 295.0593
R_{f}: 0.4 (CH₂Cl₂/MeOH 9:1)
MNR ¹H (400 MHz, CDCl₃):

| δ (ppm) | multiplicity | coupling ⁿJ (Hz) | integration | attribution |
|---|---|---|---|---|
| 7.58 | t | ³J 8.0 Hz | 1H | H-10 |
| 7.78 | t | ³J 8.0 Hz | 1H | H-9 |
| 8.09 | d | ³J 5.2 Hz | 1H | H-1 |
| 8.15 | d | ³J 8.0 Hz | 1H | H-11 |
| 8.82 | d | ³J 8.0 Hz | 1H | H-8 |
| 9.02 | d | ³J 5.2 Hz | 1H | H-2 |
| 9.07 | d | ³J 1.6 Hz | 1H | H-13 |
| 9.15 | d | ³J 1.6 Hz | 1H | H-14 |

MNR ¹³C (100 MHz, CDCl₃): 117.3 (C-1); 117.8 (C-8); 122.7
(C-11); 124.7 (C-11a); 126.2 (C-10); 131.4 (C-9); 132.1 (C-11b);
134.1 (C-3b); 139.0 (C-7a); 141.6 (C-3a); 146.0 (C-2); 146.7 (C-13);
147.4 (C-4); 147.4 (C-5); 148.7 (C-14); 157.1 (C-6).
N-oxide-canthin-6-one:

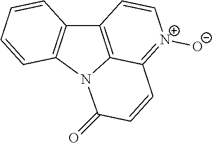

microcrystalline yellow
Melting point: 243-245° C.
IR υₘₐₓ cm⁻¹: 16S77 (—C═O)
MS (IC): m/z [M + Na]⁺ 237
MSHR (IC): for C₁₄H₉N₂O
[M + H]⁺ 237.0586, found: m/z 237.0584
R_{f}: 0.6 (CH₂Cl₂/MeOH 9:1)
MNR ¹H (400 MHz, CDCl₃):

| δ (ppm) | multiplicity | coupling ⁿJ (Hz) | integration | attribution |
|---|---|---|---|---|
| 6.92 | d | ³J 10.0 Hz | 1H | H-5 |
| 7.53 | t | ³J 8.0 Hz | 1H | H-10 |
| 7.65 | t | ³J 8.0 Hz | 1H | H-9 |
| 7.82 | d | ³J 6.6 Hz | 1H | H-1 |
| 7.99 | d | ³J 8.0 Hz | 1H | H-11 |
| 8.34 | d | ³J 6.6 Hz | 1H | H-2 |
| 8.38 | d | ³J 10.0 Hz | 1H | H-4 |
| 8.65 | d | ³J 8.0 Hz | 1H | H-8 |

MNR ¹³C (100 MHz, CDCl₃): 117.4 (C-1); 117.7 (C-8); 121.8
(C-11); 123.7 (C-11a); 126.3 (C-5); 128.4 (C-2); 129.1 (C-3b); 129.6
(C-10); 130.0 (C-9); 132.0 (C-11b); 133.0 (C-4); 134.0 (C-3b); 140.0
(C-7-a); 160.1 (C-6).
N-methyl-canthin-6-one iodide:

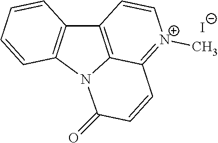

microcrystalline orange powder
Melting point: 238-241° C.
IR υₘₐₓ cm⁻¹: 1,684 (—C═O)
MS (IC): m/z [M]⁺ 235
MSHR (IC): for C₁₅H₁₁N₂O
[M]⁺ 235.0871, found: m/z 235.0873
MNR ¹H (400 MHz, δ₆ DMSO):

| δ (ppm) | multiplicity | coupling ⁿJ (Hz) | integration | attribution |
|---|---|---|---|---|
| 4.64 | s | | 3H | —Me |
| 7.41 | d | ³J 10.0 Hz | 1H | H-5 |
| 7.73 | t | ³J 7.8 Hz | 1H | H-10 |
| 7.96 | t | ³J 7.8 Hz | 1H | H-9 |
| 8.57 | m | | 3H | H-8. H-11. |
| | | | | H-4 |
| 8.90 | d | ³J 6.3 Hz | 1H | H-1 |
| 9.18 | d | ³J 6.3 Hz | 1H | H-2 |

MNR ¹³C (50 MHz, δ₆ DMSO): 44.3 (C—Me); 116.8 (C-8); 119.1
(C-1); 122.5 (C-11a); 125.7 (C-11); 127.4 (C-10); 130.2 (C-4); 130.2
(C-3a); 133.3 (C-3b); 133.7 (C-5); 134.7 (C-9); 136.1 (C-11b); 141.4
(C-7a); 142.7 (C-2); 158.0 (C-6).
N-bromoéthyl-canthin-6-one bromide:

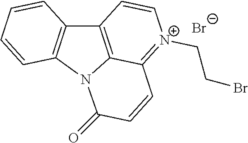

pale green microcrystalline powder
Melting point: >260° C.
IR υₘₐₓ cm⁻¹: 1,682 (—C═O)
MS (IC): m/z [M]⁺ 248
MSHR (IC): for C₁₆H₁₂N₂O
[M]⁺ 248.0950, found: m/z 248.0953
MNR ¹H (200 MHz, D₂O):

| δ (ppm) | multiplicity | coupling ⁿJ (Hz) | integration | attribution |
|---|---|---|---|---|
| 4.19 | t | ³J 5.8 Hz | 2H | H-2′ |
| 5.51 | t | ³J 5..8 Hz | 2H | H-1′ |
| 7.12 | d | ³J 10.2 Hz | 1H | H-5 |
| 7.41 | t | ³J 8.0 Hz | 1H | H-10 |
| 7.62 | t | ³J 8.0 Hz | 1H | H-9 |
| 8.06-8.16 | m | | 2H | H-8, H-11 |
| 8.27 | d | ³J 10.2 Hz | 1H | H-4 |
| 8.41 | d | ³J 6.4 Hz | 1H | H-1 |
| 8.81 | d | ³J 6.4 Hz | 1H | H-2 |

N-oxide-benzo[e]canthin-6-one

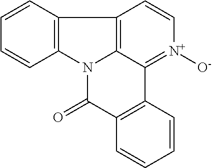

yellow powder
Melting point: >250° C.
IR νₘₐₓ cm⁻¹: 1,676 (—C═O)
MS (ES): m/z [M]⁺ 286
MSHR (ES): for C₁₈H₁₁N₂O₂
[M]⁺ 286.0742, found: m/z 286.0741
R_{f}: 0.6 (CH₂Cl₂/MeOH 9:1)
RMN ¹H (200 MHz, D₂O):

| δ (ppm) | multiplicity | coupling ⁿJ (Hz) | integration | attribution |
|---|---|---|---|---|
| 7.50 | t | ³J 8.0 Hz | 1H | H-10 |
| 7.72 | t | ³J 8.0 Hz | 1H | H-9 |
| 7.82-7.93 | m | | 2H | H-14, H-13 |
| 8.08 | d | ³J 8.0 Hz | 1H | H-11 |
| 8.19 | d | ³J 6.7 Hz | 1H | H-1 |
| 8.47-8.57 | m | | 2H | H-8, H-15 |
| 8.67 | d | ³J 6.7 Hz | 1H | H-2 |
| 9.23 | d | ³J 8.0 Hz | 1H | H-12 |

### EXAMPLE 2

### Methodology of Biological Assays

The antimycobacterial activity of canthin-6-one and some twenty canthin-6-one analog molecules was assessed on Mycobacterium bovis BCG, Mycobacterium smegmatis, Mycobacterium tuberculosis, and Mycobacterium avium.
M. bovis is very close to M. tuberculosis (Institut Pasteur, 2002); above 99% identity exists between the two genomes.

M. smegmatis is a bacterium with a rapid growth (Group IV in the Runyon classification of bacteria), with a length of 3-5 μm, sometimes curved, with a tendency to lose its acid-fast character in cultures aged over 5 days. M. smegmatis may be cultured in Middlebrook 7H10 gelose and on a Löwenstein-Jensen medium, and may grow on a MacConkey gelose without hexamethyl pararosaniline chloride. It assimilates M-inositol, D-mannitol, L-rhamnose and D-sorbitol. M. smegmatis reacts to ethambutol (5 μg/mL), and is rifampicine-(25 μg/mL) and isoniazide-resistant (10 μg/mL).

M. avium is a slow-growth bacterium, strictly aerobic and without pigmentation. M. avium may be cultured on a Löwenstein-Jensen medium or a Coletsos medium.

All molecules under test were prepared by chemical synthesis.
The antimycobacterial properties of canthin-6-one and its analogs were shown by assessing the inhibitory activity on Mycobacterium bovis BCG, Mycobacterium smegmatis, Mycobacterium tuberculosis, and Mycobacterium avium, as expressed with the MIC (Minimum Inhibitory Concentration).

In Vitro Assay on Mycobacterium bovis BCG

The liquid culture medium which is used is a 7H9 medium (Middlebrook 7H9 Difco) to which is added Tween 80 and OADC (Oleic acid, Albumin Fraction V, Dextrose and Catalase) (complement for Middlebrook OADC culture medium).

Assays are made in a sterile atmosphere with MICROTEST™ flat bottom 96 well plates. The attenuated Mycobacterium bovis strain which is used is Strain 040812 from Institut Pasteur, 180 μL of a BCG suspension with 1.10⁵ cfu (colony forming units)/mL are poured into each cup (except for the outer cups, which are filled with sterile water).

The molecules to be assayed are diluted in the culture medium (for the hardly soluble molecules dilutions are made with a maximum concentration of 1% DMSO (dimethyl sulfoxide)). 20 μL of each dilution are poured into the corresponding cups. All test are carried out three times.

The various controls as left on each plate are the following:
Positive control BCG alone in the culture medium (200 μL);
Negative control culture medium alone (200 μL);
Control with solvent: BCG (198 μL) with DMSO (10 μL).
Reference antibiotics which are used are isoniazide and ethambutol. The plates are then wrapped in Parafilm® and left in an incubator during 6 days at 37° C.
   A first reading of the results is made with a reverse microscope, at the smallest magnification. The MIC corresponds to the last concentration for which there is no bacterial veil.

Results are confirmed by MTT coloration (methylthiazoyltetrazolium bromide). The MTT is prepared extemporaneously in a 5 mg/mL buffered phosphate solution, and 50 μL of this solution are added in each well. The plates are then incubated during 4 hours at 37° C. and sheltered from light. After this period 50 μL DMSO/ethanol 50/50 (v/v) are added and the plates are brought back to 37° C. during 24 hrs. Finally the last step is a reading of the optical density (OD) at 570 nm. The MIC may be determined by observing a notable increase in the OD, corresponding to the presence of living bacterial colonies.

The MIC of isoniazide is determined by this method

MIC=0.10 μg/mL

and for ethambutol:

MIC=5 μg/mL

TABLE
In vitro activity of canthin-6-one and
analogs on Mycobacterium bovis BCG

| | Minimal Inhibitory | |
|---|---|---|
| | Concentration | |
| MOLECULES | MIC (μg/mL) | MIC (μmol/L) |
| 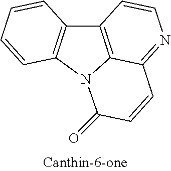 | 7.5 | 34.1 |
| 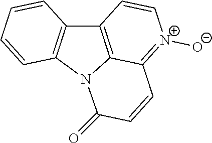 | 2.5 < CMI < 5* | 10.6 < CMI < 21.2* |
| 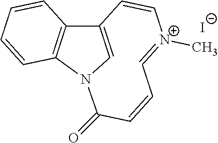 | 7.5 | 31.9 |
| 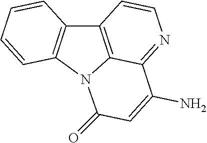 | 10 | 42.5 |
| 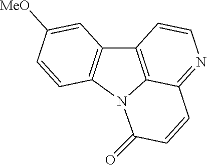 | 7.5 | 30.0 |
| 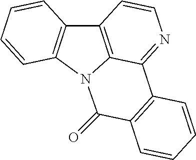 | 2.5 < CMI < 5* | 9.2 < CMI < 18.4* |
| 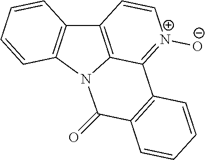 | 10 | 34.8 |
| 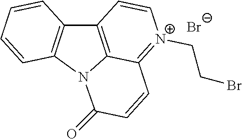 | 1 < CMI < 5* | 3.0 < CMI < 15.3* |
| 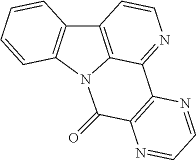 | 10 | 36.8 |
| 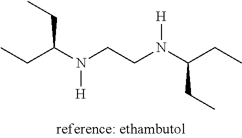 | 5 | 24.5 |
| 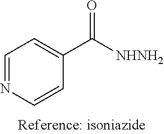 | 0.1 | 0.73 |

*In bold type, equivalent activity to that of ethambutol.

As may be deduced from the Table of results, several molecules which were tested have shown Minimum Inhibitory Concentrations (MIC in μg) which were equivalent to that of antituberculous reference molecules, viz. ethambutol or pyrazinamide, with MICs between 1 to 5 μg/mL.

Canthin-5-one and its analogs have shown in the above-described activity assays a surprising efficiency against Mycobacterium bovis BCG.

The other compounds have shown appreciable anti-mycobacterial properties.

In Vitro Assays on Mycobacterium smegmatis

The mycobacteria inoculum, 2-3.4×10⁴ cfu (Colony Forming Units)/mL is prepared from the mc²155 strain of M. smegmatis and cultured in Dubos medium (Difco, USA) to which is added 10% OADC (Oleic acid, Albumin Fraction V, Dextrose and Catalase), 100 μl of the inoculum are poured into each wells.

In a first step the molecules which were the most active on M. bovis(canthin-6-one, N-oxide-canthin-6-one, benzo[e]canthin-6-one and N-bromoethyl-canthin-6-one bromide) were tested, and in a second step all canthin-6-one analogs were tested.

All molecules to be tested are prepared in dimethylsulfoxide (DMSO) at a concentration of 50 mg/ml and frozen until use.

20 μl of each dilution for each molecule are prepared in 100 μl of the Dubos culture medium (Difco, USA), and then distributed into 96 well plates, at a concentration between 100-0.75 μg/ml.

Control wells containing DMSO alone and ethambutol (Etibi®, Myambutol®) or ofloxacine (synthesis antibiotic belonging to the fluoroquinolone family) are included at a concentration between 1 μg/l-1 ng/ml.

The plates are covered, sealed and incubated at 37° C. during 48 hours.

Minimal Inhibitory Concentrations (MIC) are determined using the resazurin coloration microdilution method. The powdery resazurin salt (Sigma) is prepared at a concentration of 0.01% (weight/vol) in distilled water, and the solution is then sterilized by 0.22 μm membrane filtration, and kept at 4° C. during one week.

Into the plates as incubated during 48 hours, 30 μl of a resazurin solution are added in each well. The plates are then incubated at 37° C. during 24 hours. The colour variation from blue to pink is indicative of a decrease in resazurin which corresponds to the development of the bacterial colony. The MIC is determined as being the lowest concentration to prevent a colour change. The optical density of each well is measured at 530-630 nm using a micro-plate reader, The MIC is determined by drawing the dose-optical density response curve.

The ofloxacin MIC is determined with this method

CMI=1 μg/ml,

and for ethambutol

CMI=0.5 μg/ml.

Several molecules have shown an efficiency which is equivalent to that of antituberculous reference molecules.

Canthin-6-one and its analogs have shown themselves to be efficient, in the hereabove-described activity tests, against Mycobacterium smegmatis.

In Vitro Tests on Mycobacterium tuberculosis

Slow growing mycobacteria such as M. tuberculosis H37Rv are cultured in Löwenstein-Jensen medium (Difco Laboratories, USA).

In a first step the molecules which were the most active on M. bovis and which are hereabove mentioned have been tested, and in a second step all canthin-6-one analogs have been analyzed.

The molecules to be tested are diluted in a culture medium—for scarcely soluble molecules dilutions are made with a maximum concentration of 1% DMSO (dimethylsulfoxide). All tests are repeated three times.

The Minimum Inhibitory Concentrations are determined in a 7H11 agar medium to which is added 10% OADC (Oleic acid, Albumin Fraction V, Dextrose and Catalase) containing 10³-10⁵ cfu (colony forming unit)/ml.

The mycobacterial colonies are enumerated after 21-30 days incubation at 37° C. The Minimum Inhibitory Concentration is determined as being the smallest concentration of canthin-6-one derivatives reducing their growth by at least 1% as compared with an untreated control colony.

The MIC of rifampicin (antituberculous antibiotic belonging to the rifamycin family) is:

MIC=0.1 μg/ml

and for ofloxacin:

MIC=1 μg/ml.

Several molecules have shown an efficiency which is equivalent to that of antituberculous reference molecules.

Canthin-6-one and its analogs have shown, in the hereabove-described activity tests, to be efficient against Mycobacterium tuberculosis.

In Vitro Tests on Mycobacterium avium

Slow growing mycobacteria such as M. avium 101 are cultured in Löwenstein-Jensen medium (Difco Laboratories, USA).

In a first step the molecules which are the most active on M. bovis and which are hereabove cited have been tested, and in a second step all canthin-6-one analogs have been analyzed.

The molecules to be tested are diluted in a culture medium—for the scarsely soluble molecules dilutions are made with a maximum concentration of 1% DMSO (dimethylsulfoxide). All tests are repeated three times.

The Minimum Inhibitory Concentrations are determined in a 7H11 agar medium to which is added 10% OADC (Oleic acid, Albumin Fraction V, Dextrose and Catalase) containing 10³-10⁵ cfu (colony forming units)/ml.

The mycobacterial colonies are enumerated after 21-30 days incubation at 37° C. The Minimal Inhibitory Concentration is determined as being the smallest canthin-6-one derivatives concentration which reduced growth by at least 1%, as compared with an untreated control colony.

The MIC of isoniazide (antituberculous derivative of isonicotinic acid) is:

MIC=0.1 μg/ml

and for ofloxacin

MIC=6.2 μg/ml.

Several molecules have shown an efficiency which is equivalent to that of antituberculous reference molecules.

Canthin-6-one and its analogs have shown, in activity tests such as hereabove described, to be efficient against Mycobacterium avium.

### EXAMPLE 3

### In Vivo Tests on Mycobacterium bovis BCG

Test Animals:

Animals used in these tests are C57B1/6 female mice.

Infection:

200 μL of a BCG suspension at a concentration of 5×10⁵ VU/mL (VU: viable units) in a physiological saline solution are inoculated to the various mice after 7 days housing.

Treatment:

Treatment begins 15 days after BCG inoculation and for a duration of 5 days (once a day). All tested molecules (canthin-6-one and its analogs) are suspended in a physiological saline solution (with 1% DMSO), and 200 μL are given to the mice intraperitoneally.

The reference product which is used is isoniazide at a dose of 25 mg/kg/day, and the molecules are tested at a dose of 20 mg/kg/day.

Parameters Under Study:

The antituberculous activity is assessed by counting the number of bacteria at the spleen, liver and lungs levels.

These organs are removed at the end of treatment after killing the mice. The organs are later comminuted in a Sauton buffer and laid onto 7H11 geloses to which OADC is added (Oleic acid, Albumin Fraction V, Dextrose and catalase).

Counting on each box is carried out 2-3 weeks after incubation at 37° C.

Several molecules have shown an efficiency which is equivalent to that of isoniazide.

Canthin-6-one and its analogs have shown, in the hereabove described in vivo activity tests, to be efficient against Mycobacterium bovis BOG.

1-16. (canceled)

17. A method for the treatment or prevention of pathologies which are linked to, or caused by, mycobacteria, comprising the administration in a patient in need thereof of at least one of the following Formula (I) compounds: 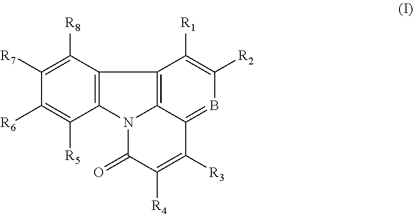
wherein:
B represents:
a nitrogen atom, or
a N-oxide N⁺—O⁻ group, or
a group having the formula N⁺—R or 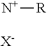
R represents an alkyl group, which may be linear, branched or cyclic, saturated or unsaturated, comprising from 1 to 12 carbon atoms, which may be substituted, for instance by at least one halogen atom, and X⁻ represents an anion which may be chosen among mineral or organic anions.
R₁, R₂, R₃, R₄, R₅, R₆R₇ and R₈ independently represent:
a hydrogen atom,
an alkyl group, which may be linear, branched or cyclic, saturated or unsaturated, comprising from 1 to 12 carbon atoms,
a halogen atom, chosen among chlorine, fluorine, bromine and iodine,
a halogenoalkyl group, wherein the alkyl chain is linear, branched or cyclic, saturated or unsaturated, comprising from 1 to 12 carbon atoms, and wherein the halogen atom(s) are chosen among fluorine, chlorine, bromine and iodine,
a hydroxyl group,
a nitro —NO group,
a —CN group,
a —SH group,
a carboxylic acid —COOH group,
an amide —CONH₂ group,
an amine —NH₂ group,
an alcoxy —ORₐ group, wherein Rₐ represents an alkyl group, which may be linear, branched or cyclic, saturated or unsaturated, comprising from 1 to 12 carbon atoms,
an alkylester —COOR_{b}, group, wherein R_{b} represents an alkyl group, which may be linear, branched or cyclic, saturated or unsaturated, comprising from 1 to 12 carbon atoms,
a secondary (—NHCOR_{c}) or tertiary (—N(COR_{d})COR_{c}) alkylamide group, wherein R_{c} and R_{d} independently represent an alkyl group, which may be linear, branched or cyclic, saturated or unsaturated, comprising from 1 to 12 carbon atoms,
a secondary (—NHRₑ) or tertiary (-NrₑR_{f}) alkylamine group, wherein Rₑ and R_{f} independently represent an alkyl group, which may be linear, branched or cyclic, saturated or unsaturated, comprising from 1 to 12 carbon atoms,
an alkylthio (—Sr_{g}) group, wherein R_{g} represents an ankyl group, which may be linear, branched or cyclic, saturated or unsaturated, comprising from 1 to 12 carbon atoms,
a C₂-C₆ heterocyclic group, comprising from 1 to 4 heteroatoms chosen among sulphur, nitrogen and oxygen,
a —SO₂—NRₕRᵢ group or a -Nrₕ-SO₂—Rᵢ group, wherein Rₕ and Rᵢ independently represent an alkyl group, which may be linear, branched or cyclic, saturated or unsaturated, comprising from 1 to 12 carbon atoms,
the groups R₁, R₂, R₃, R₄, R₅, R₆, R₇ and R₈ may also form the following intramolecular cyclisations:
1/ cyclisation between R¹ and R² and/or
2/ cyclisation between R³ and R⁴ and/or
3/ cyclisation between R⁵ and R⁶ and/or
4/ cyclisation between R⁶ and R⁷ and/or
5/ cyclisation between R⁷ and R⁸,
6/ cyclisation between R³ and B, notably when B represents N⁺—R,
7/ cyclisation between R² and B, notably when B represents N⁺—R,
said thus formed cycles being notably aromatic cycles comprising from 5 to 30 carbon atoms, and which may also comprise at least one heteroatom chosen among: O, N, S, the aromatic cycles being for instance chosen among benzene, naphthalene, pyridine, pyrrole, thiophene, furan, pyrazine,
said cycles may be substituted by
an hydrogen atom,
an alkyl group, which may be linear, branched or cyclic, saturated or unsaturated, comprising from 1 to 12 carbon atoms,
a halogen atom, which is chosen among chlorine, fluorine, bromine and iodine,
a halogenoalkyl group, wherein the alkyl chain is linear, branched or cyclic, saturated or unsaturated, comprising from 1 to 12 carbon atoms, and wherein the halogen atom(s) are chosen among fluorine, chlorine, bromine and iodine,
a hydroxyl group,
a nitro —NO group,
a cyano —ON group,
a —SH group,
a carboxylic acid —COOH group,
an amide —CONH₂ group,
an amine —NH₂ group,
an alcoxy —ORₐ group, wherein Rₐ represents an alkyl group, which may be linear, branched or cyclic, saturated or unsaturated, comprising from 1 to 12 carbon atoms,
an alkylester —COOR_{b} group, wherein R_{b} represents an alkyl group, which may be linear, branched or cyclic, saturated or unsaturated, comprising from 1 to 12 carbon atoms,
a secondary (—NHCOR_{c}) or tertiary (—N(COR_{d})COR_{c}) alkylamide group, wherein R_{c} and R_{d} independently represent an alkyl group, which may be linear, branched or cyclic, saturated or unsaturated, comprising from 1 to 12 carbon atoms,
a secondary (—NHRₑ) or tertiary (—NRₑR_{f}) alkylamine group, wherein Rₑ and R_{f} independently represent an alkyl group, which may be linear, branched or cyclic, saturated or unsaturated, comprising from 1 to 12 carbon atoms,
an alkylthio (—SR_{g}) group, wherein R_{g} represents an alkyl group, which may be linear, branched or cyclic, saturated or unsaturated, comprising from 1 to 12 carbon atoms,
a C₂-C₆ heterocyclic group comprising from 1 to 4 heteroatoms chosen among sulphur, nitrogen and oxygen,
a —SO₂—NRₕRᵢ group or a —NRₕ—SO₂—Rᵢ group, wherein Rₕ and Rᵢ independently represent an alkyl group, which may be linear, branched or cyclic, saturated or unsaturated, comprising from 1 to 12 carbon atoms from 1 to 6 carbon atoms.

18. The method according to claim 17, wherein said alkyl group comprises from 1 to 6 carbon atoms.

19. The method according to claim 17, characterized in that the mycobacteria are chosen among the group comprising Mycobacterium tuberculosis, Mycobacterium bovis, Mycobacterium smegmatis, Mycobacterium africanum, Mycobacterium leprae, Mycobacterium kansasii, Mycobacterium xenopi, Mycobacterium avium intracellulaire, Mycobacterium scrofulaceum, Mycobacterium marinum, Mycobacterium fortuitum, Mycobacterium chelonei, Mycobacterium ulcerans and Mycobacterium abcessus, notably Mycobacterium tuberculosis and Mycobacterium bovis.

20. The method according to claim 17, characterized in that the pathologies which are linked to, or caused by, mycobacteria, are chosen among:
Tuberculosis,
Buruli's ulcer,
Nosocomial infections,
Leprosy,
Cutaneous infections,
Soft tissue infections,
Osteomyelites,
Localized abscesses, and
Lipoid pneumoniae.

21. The method according to claim 20, wherein Tuberculosis, is chosen among:
Tuberculosis of the respiratory tract (pulmonary tuberculosis, tuberculous lymphadenitis, larynx, trachea and bronchi tuberculosis, tuberculous pleurisy and tuberculous primary infection of the respiratory tract),
Tuberculosis of the nervous system (tuberculous meningitis, tuberculous polyneuritis, tuberculous myelitis),
Tuberculosis of the bone and joints,
Tuberculosis of the urogenital tract,
Peripheral tuberculous adenopathy,
Tuberculosis of the intestine, the peritoneum and the mesenteric glands
Tuberculosis of the skin and the subcutaneous cellular tissue
Tuberculosis of the eye and ear
Tuberculosis of the adrenal glands
Acute miliary tuberculosis.

22. The method according to claim 17, characterized in that X⁻ is chosen among: Cl⁻, Br⁻, I⁻, PO₃⁻, NO₃⁻, acetate, oxalate, tartrate, succinate, maleate, fumarate, gluconate, citrate, malate, ascorbate and benzoate.

23. The method according to claim 17, characterized in that B represents a nitrogen atom, and the compounds correspond to the following Formula (II):

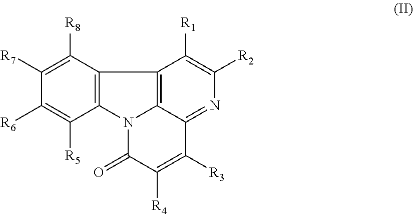

24. The method according to claim 17, characterized in that B represents a N-oxide NO group, and the compounds correspond to the following Formula (III):

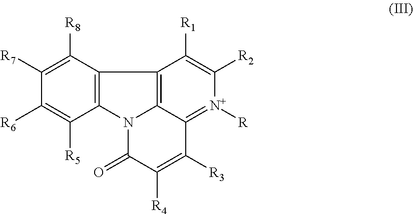

25. The method according to claim 17, characterized in that B represents a group having Formula 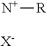
wherein X⁻ and R are as previously defined, and the
compounds correspond to the following Formula (IV): 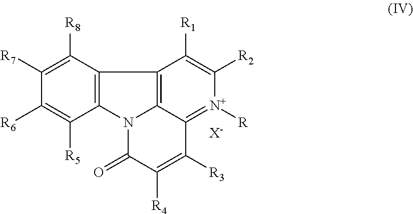
wherein R notably represents a methyl or ethyl group, for instance substituted by at least one halogen atom.

26. The method according to claim 25, wherein said halogen atom is chosen among F, Cl, Br or I.

27. The method according to claim 17, characterized in that R₃ and R₄ form a benzene cycle between them, and the compounds correspond to the following Formula (I-1):

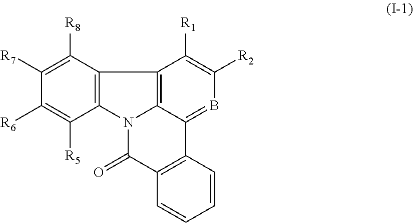

28. The method according to claim 17, characterized in that the compounds are chosen among: 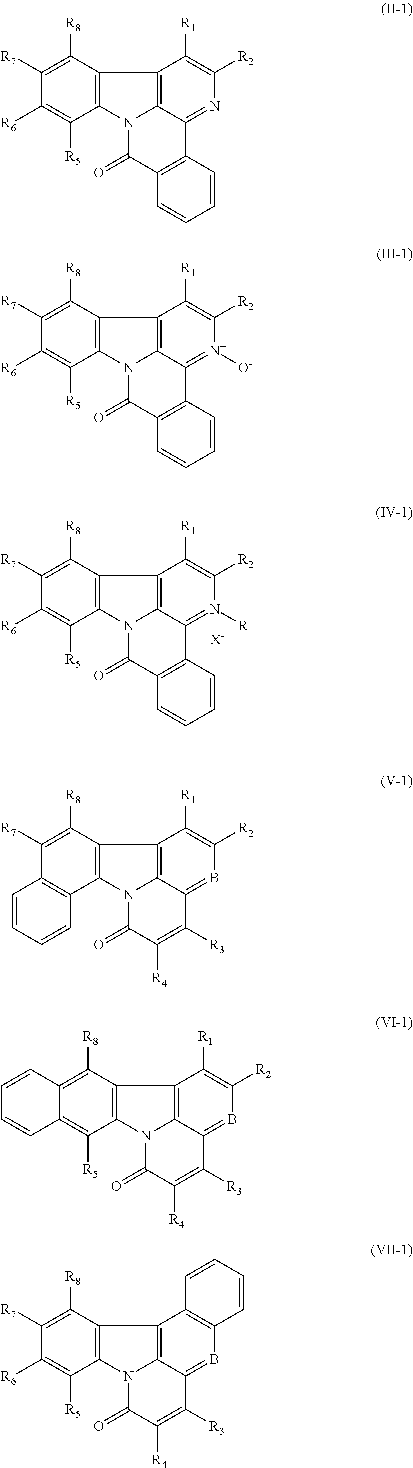 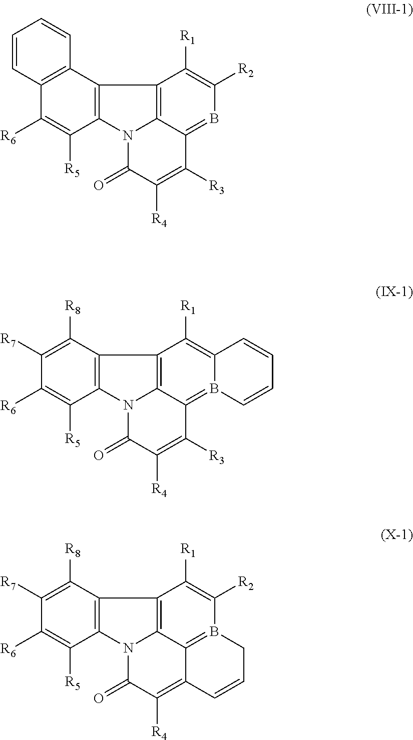
wherein R₁ to R₈, B and X⁻ have the meanings as given in claim from 1.

29. The method according to claim 17, characterized in that at least one of the radicals R₅-R₈ and notably R₆ represents a halogen atom, notably a fluorine atom.

30. The method according to claim 17, characterized in that B represents a nitrogen atom, and in that R₁, R₂, R₃, R₄, R₅, R₆, R₇ d R₈ represent a hydrogen atom, said compound corresponding to the following formula:

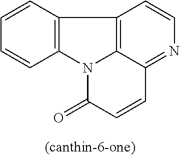

31. The method according to claim 17, characterized in that the Formula (I) compound is chosen among one of the following compounds:

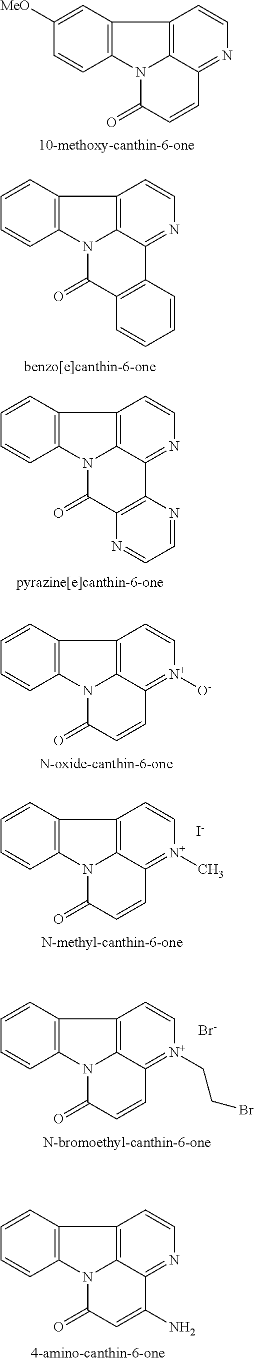

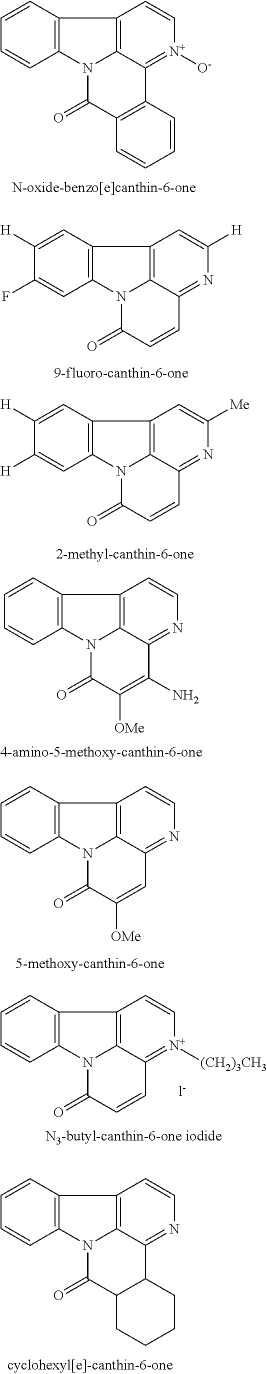

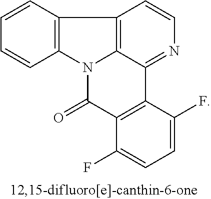

32. The method according to claim 17, characterized in that the Formula (I) compound is given at a dose of from 0.01 mg/kg/day to 100 mg/kg/day.

33. The method according to claim 32, characterized in that the Formula (I) compound is given at a dose of from 0.1 to 50 mg/kg/day.

34. The method according to claim 32, characterized in that the Formula (I) compound is given at a dose of from 1 to 20 mg/kg/day.

35. The method according to claim 17, characterized in that the Formula (I) compound is given orally.

36. A compound corresponding to the following Formula:

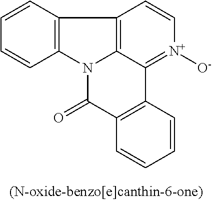

37. A pharmaceutical composition comprising as an active substance the compound according to claim 36, in association with a pharmaceutically acceptable excipient.
